(19) United States
(12) Patent Application Publication
(10) Pub. No.: US 2020/0188115 A1
(43) Pub. Date: Jun. 18, 2020

(54) HEART VALVE REPAIR TOOL AND TECHNIQUES

(71) Applicant: Medtronic Vascular, Inc., Santa Rosa, CA (US)

(72) Inventors: Caitlin Dorff, Santa Rosa, CA (US); Fatemeh Fatemi Far, Santa Rosa, CA (US); Matthew E. Genovese, Windsor, CA (US); Emily Grimm, Petaluma, CA (US); Olivia Metcalf, Santa Rosa, CA (US); Karan Punga, San Rafael, CA (US)

(21) Appl. No.: 16/710,999

(22) Filed: Dec. 11, 2019

Related U.S. Application Data
(60) Provisional application No. 62/779,208, filed on Dec. 13, 2018.

(51) Int. Cl. A61F 2/24

(52) U.S. Cl. CPC A61F 2/2466; A61F 2220/0016; A61F 2/2457

(57) ABSTRACT

A catheter tool system for implanting an artificial chordal attachment to the mitral valve of a heart includes a first catheter defining a first lumen; an anchor configured be located within the first lumen and to attach to a tissue of a left ventricle of a heart; a plug configured to be located within the first lumen and to attach to a leaflet of a mitral valve of the heart; a first line connecting the anchor to the plug within the first lumen; a second catheter defining a second lumen; a stabilizer member configured to be located within the second lumen and to extend out of the second lumen to stabilize the leaflet of the mitral valve relative to the second catheter; and a second line connected to the stabilizer member within the second lumen.

This application claims the benefit of U.S. Provisional Patent Application Ser. No. 62/779,208, filed on Dec. 13, 2018, the entire content of which is incorporated by reference herein.

### TECHNICAL FIELD

This disclosure relates to heart valve repair, such as mitral valve repair.

### BACKGROUND

Some patient conditions can produce valvular insufficiency or regurgitation. Valvular insufficiency or regurgitation occurs when a valve in a heart of a subject does not close completely, allowing blood to flow backwards (e.g., from the left ventricle to the left atrium), which may adversely impact the functionality of the heart.

The mitral valve includes two leaflets (anterior and posterior) attached to an annulus (e.g., a fibrous ring). In a healthy heart, the mitral valve leaflets close during contraction of the left ventricle and prevent blood from flowing back into the left atrium. Mitral valve regurgitation is a condition in which the leaflets of a mitral valve of a subject do not coapt properly and, as a result, blood regurgitates back into the left atrium from the left ventricle. The regurgitation of blood back into the left atrium may result in a reduced ejection volume from the left ventricle, causing the heart of the subject to work relatively hard to supply the desirable volume of blood to the body. Mitral regurgitation may occur because of different patient conditions. For example, secondary mitral regurgitation, also referred to as functional mitral regurgitation, may occur when a left ventricle dilates and causes dilation of the mitral annulus of a subject.

### SUMMARY

In some examples, the disclosure describes tools and techniques that may be used to replace or add a chordal attachment (also referred to a chordal tendinea) between a leaflet of a valve and adjacent tissue. For example, example tools and techniques of the disclosure may be used to replace or add chordal attachment(s) between a leaflet of the mitral valve and the papillary muscle or other tissue of the left ventricle of the heart.

In some examples, a tool includes a first catheter and second catheter, e.g., which are both contained and extend adjacent to each other within a lumen of an outer catheter, configured to be inserted into the left atrium of a heart of a patient, e.g., via transfemoral and trans-septal pathway. The first catheter may define a first lumen that contains an anchor and a plug attached to each other via a first line. The first catheter may be configured to puncture a leaflet of the mitral valve while the second catheter stabilizes the leaflet. To create the chordal attachment, the anchor may be attached to a tissue of the left ventricle and the plug may be configured to be secured within the puncture in the leaflet. The line connecting the anchor to the plug may be adjustable to provide for a desired length between the anchor and the plug while connected to the tissue of the left ventricle and leaflet, respectively.

In some examples, the disclosure is directed a catheter tool system comprising a first catheter defining a first lumen; an anchor configured be located within the first lumen and to attach to a tissue of a left ventricle of a heart; a plug configured to be located within the first lumen and to attach to a leaflet of a mitral valve of the heart; a first line connecting the anchor to the plug within the first lumen; a second catheter defining a second lumen; a stabilizer member configured to be located within the second lumen and to extend out of the second lumen to stabilize the leaflet of the mitral valve relative to the second catheter; and a second line connected to the stabilizer member within the second lumen.

In another example, the disclosure is directed to a catheter tool system comprising a first catheter defining a first lumen; an anchor configured be located within the first lumen and to attach to a tissue of a left ventricle of a heart; a second catheter within the first lumen and defining a second lumen; a stabilizer member configured to be located within the second lumen and to extend out of an opening in the first catheter to stabilize the leaflet of the mitral valve relative to the second catheter; a sharp tip member within the first lumen pointing away from a distal end of the first catheter; and a line connected to the anchor and the sharp tip member.

In another example, the disclosure is directed to a method comprising forming an artificial chordal attachment between a leaflet of a heart valve and an adjacent tissue using the described tool system.

The details of one or more examples are set forth in the accompanying drawings and the description below. Other features, objects, and advantages of examples according to this disclosure will be apparent from the description and drawings, and from the claims.

### BRIEF DESCRIPTION OF THE DRAWINGS

FIGS. 1A and 1B are schematic cross-sectional views of an example human heart.

FIG. 2 is a conceptual schematic diagram illustrating an example tool system that may be used, e.g., to add an artificial chordal attachment between a leaflet of a valve of the heart and tissue of the left ventricle, such as the papillary muscle of the left ventricle.

FIG. 3 is a conceptual schematic diagram illustrating a magnified view of the portion of tool system illustrated in FIG. 2.

FIG. 4 is a conceptual schematic diagram illustrating another magnified view of the portion of tool system illustrated in FIG. 2.

FIG. 5 is a conceptual schematic diagram illustrating another magnified view of the portion of tool system illustrated in FIG. 2.

FIGS. 6A-6C is a conceptual schematic diagram illustrating an example foot design of the stabilizer member of the portion of tool system illustrated in FIG. 2.

FIGS. 7A and 7B are conceptual schematic diagrams illustrating alternative example of a foot design of the stabilizer member of the portion of tool system illustrated in FIG. 2.

FIGS. 8A-8C are conceptual schematic diagrams illustrating the deployment of the petal design of an example stabilizer member.

FIG. 9 is a conceptual diagram illustrating an example top view of a stabilizer of an example second catheter deployed to stabilize the leaflet by engaging the tissue of leaflet.

FIG. 10 is a conceptual diagram illustrating an example hole puncture catheter along with a portion of tool system illustrated in FIG. 2.

FIGS. 11A and 11B are conceptual diagrams illustrating an example of hole puncture catheter with pointed tip and sharpened ring, respectively, attachment at the distal end.

FIG. 12 is a conceptual diagram illustrating the position of anchor within the left ventricle and the position of a plug near the leaflet surface.

FIGS. 13A and 13B are conceptual diagrams illustrating details of an example plug.

FIGS. 14A-14E are conceptual diagrams illustrating deployment of an example plug.

FIGS. 15A and 15B are conceptual diagrams illustrating the adjustment of a first line to adjust the length between an anchor and a plug.

FIGS. 16, 17A-17C, 18, and 19 are conceptual diagrams illustrating example anchors including possible wire shapes and connection mechanisms.

FIG. 20 is a conceptual diagram illustrating an example of a tool system used for an anchor first process for implanting an artificial chordal attachment between a leaflet of the mitral valve and tissue of the left ventricle.

FIGS. 21A and 21B are conceptual diagrams illustrating examples of anchors that may be used to anchor an artificial chordal attachment to a tissue of the left ventricle in accordance with some examples of the disclosure.

FIG. 22 is a conceptual diagram illustrating example shapes of anchors.

FIGS. 23 and 24 are conceptual diagrams illustrating example designs of a stabilizer member.

FIG. 25 is a conceptual diagram illustrating other example configurations for a plug and a first line.

FIG. 26A-26C are flow diagrams illustrating an example technique of the disclosure.

FIGS. 27A-27D are conceptual diagrams illustrating an example process for implanting an artificial chordal attachment between a leaflet of the mitral valve and tissue of the left ventricle using the described tool system.

FIG. 28 is a conceptual diagram illustrating an example of a tool system used for an anchor first process for implanting an artificial chordal attachment between a leaflet of the mitral valve and tissue of the left ventricle.

### DETAILED DESCRIPTION

This disclosure describes devices, systems, and techniques for repairing a heart valve, such as, but not limited to, a mitral valve. As described above, the disclosure describes tools and techniques that may be used to replace or add a chordal attachment (also referred to a chordal tendinea) between a leaflet of a heart valve and adjacent tissue. For example, example tools and techniques of the disclosure may be used to replace or add chordal attachment(s) between a leaflet of the mitral valve and the papillary muscle or other tissue of the left ventricle of the heart. Such a procedure may be carried out to treat one or more patient conditions, such as, degenerative mitral valve (DMR) disease. While examples are described primarily with regard to the creation of an attachment between a leaflet of the mitral valve and tissue of the left ventricle, such examples and tool may be used to create attachment between leaflets and adjacent tissue of other valves of a heart of a patient.

In some examples, a tool includes a first catheter and second catheter, e.g., which are both contained and extend adjacent to each other within a lumen of an outer catheter, that are configured to be inserted into the left atrium of the heart of a patient, e.g., via trans-femoral and trans-septal pathway. The first catheter may define a first lumen that contains an anchor and a plug attached to each other via a first line. The first catheter (also referred to as a puncture catheter) may be configured to puncture a leaflet of the mitral valve while the second catheter (also referred to as a stabilizer catheter) stabilizes the leaflet. To create the chordal attachment, the anchor may be attached to a tissue of the left ventricle and the plug may be configured to be secured within the puncture in the leaflet. The line connecting the anchor to the plug may be adjustable to provide for a desired length between the anchor and the plug while connected to the tissue of the left ventricle and leaflet, respectively.

As described herein, the tool may be used to replace and/or add a chordal attachment using a leaflet first or anchor first process. For example, in a leaflet first process, a leaflet of a mitral valve may be punctured from the atrial side by the first catheter of the tool prior to anchoring in the left ventricle. The second catheter may be used to stabilize the leaflet, e.g., by temporarily connecting or applying a stabilizing force to the leaflet, while the first catheter punctures the leaflet. An anchor may be retracted or otherwise located within the first catheter while the first catheter punctures and then passes through the leaflet via the puncture into the left ventricle. The anchor may then be attached to the tissue of the left ventricle and the first catheter may be withdrawn back into the left atrium through the puncture. A plug contained within the first catheter and attached to the anchor with a connection line may be placed within the puncture to connect the leaflet to the anchor. The second stabilizing catheter may be disengaged from the leaflet and the tool may be removed from the body of the patient leaving the artificial chordae in place within the left ventricle.

As another example, in an anchor first process, the anchor may be attached to tissue of the left ventricle, and then the leaflet of the mitral valve is punctured and attached to the artificial chordae from the ventricular side.

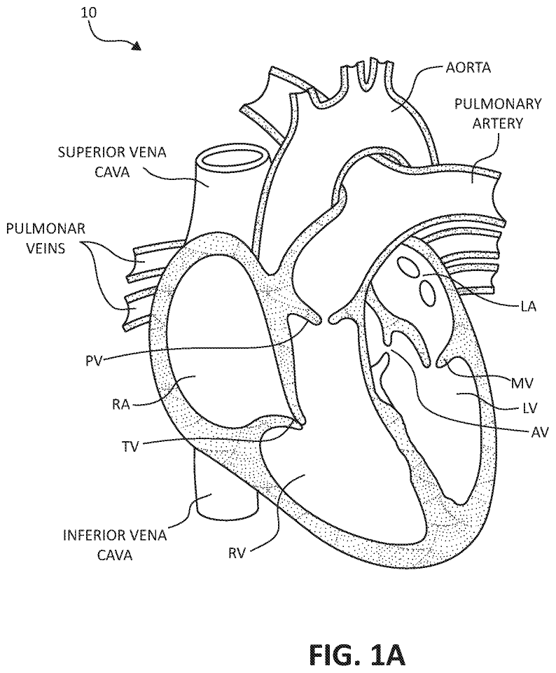

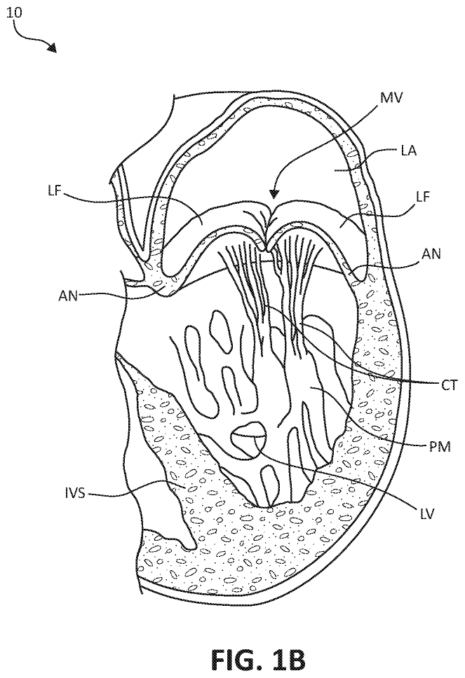

FIGS. 1A and 1B are schematic cross-sectional views of an example human heart 10. The human heart 10 is a four chambered, muscular organ that provides blood circulation through the body during a cardiac cycle. The four main chambers include the right atrium (RA) and right ventricle (RV) which supplies the pulmonary circulation, and the left atrium (LA) and left ventricle (LV) which supplies oxygenated blood received from the lungs to the remaining body. To ensure that blood flows in one direction through the heart, atrioventricular valves (tricuspid valve (TV) and mitral valves (MV) are present between the junctions of the atrium and the ventricles, and semi-lunar valves (pulmonary valve (PV) and aortic valve (AV)) govern the exits of the ventricles leading to the lungs and the rest of the body. These valves contain leaflets (LF) or cusps that open and shut in response to blood pressure changes caused by the contraction and relaxation of the heart chambers. FIG. 1B is a schematic sectional illustration of a left ventricle LV of heart 10 showing anatomical structures and a native mitral valve MV.

The left atrium LA receives oxygenated blood from the lungs via the pulmonary veins and pumps the oxygenated blood through the mitral valve MV and into the left ventricle LV during ventricular diastole. The left ventricle LV contracts during systole and blood flows outwardly through the aortic valve AV, into the aorta and to the remainder of the body. In a healthy heart, the leaflets LF of the native mitral valve MV meet evenly at the free edges or “coapt” to close and prevent back flow of blood into the left atrium LA during contraction of the left ventricle LV. The tissue of the leaflets LF attach to the surrounding heart structure via a dense fibrous ring of connective tissue called an annulus AN. The flexible tissue of the leaflets LF of the native mitral valve MV are connected to papillary muscles PM, which extend upwardly from the lower wall of the left ventricle LV and the interventricular septum IVS, via branching tendons called chordae tendinae CT. As described herein, example tools and techniques of this disclosure may be used to replace and/or add artificial chordae tendinae, e.g., in patients with DMR.

Mitral valve regurgitation is a condition in which the leaflets of a mitral valve of a subject do not coapt properly and, as a result, blood regurgitates back into the left atrium LA from the left ventricle LV. The regurgitation of blood back into the left atrium LA may result in a reduced ejection volume from the left ventricle LV, causing the heart of the subject to work relatively hard to supply the desirable volume of blood to the body. Mitral regurgitation may occur because of one or more patient conditions. For example, secondary mitral regurgitation, also referred to as functional mitral regurgitation, may occur when the left ventricle LV dilates and causes dilation of the mitral annulus of a subject. The leaflets LF of the valves may move apart as a result of the dilation of the left ventricle LV, which may adversely impact the ability of the leaflets to properly coapt.

In addition to or instead of being caused by dilation of the left ventricle LV, mitral valve regurgitation (or other valve regurgitation) may be caused by calcified plaque buildup in heart 10. For example, the leaflets LF of the valves (e.g., aortic valve AV or mitral valve MV) may harden and may not sufficiently coapt or meet, such that regurgitation may occur where the valve does not close completely, allowing blood to flow backwards (e.g., from the left ventricle LV to the left atrium LA). The left side of heart 10 (e.g., mitral valve MV and aortic valve AV) can be more likely to become calcified because of the higher pressures generated.

The medical devices, systems, and techniques described herein may be used to repair a valve of heart 10 via a minimally invasive medical procedure, e.g., via a transcatheter, trans-septal medical procedure that is less invasive than open heart surgery. The procedure may also be a trans-femoral procedure. While open heart surgeries, such as annuloplasty performed via open heart surgery, may have positive outcomes, a more minimally invasive medical procedure may also have positive outcomes while also being associated with a shorter recovery time for some patients compared to open heart surgery.

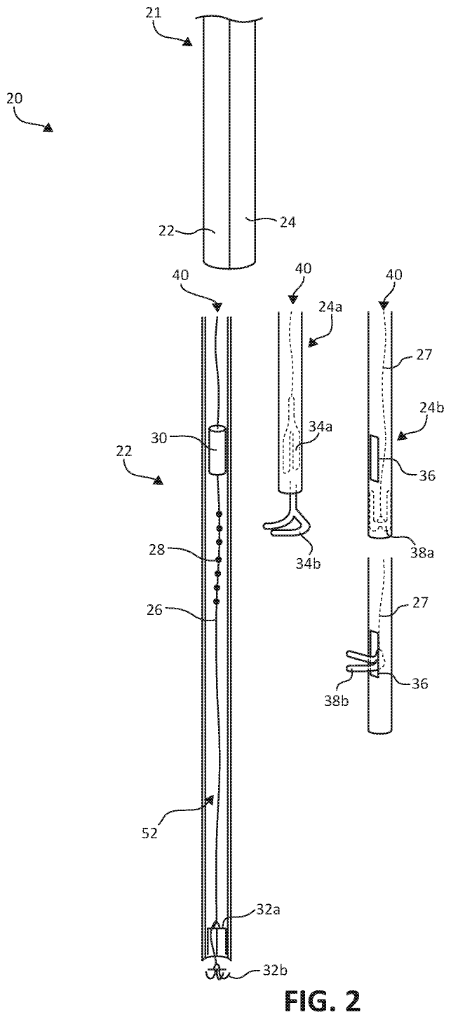

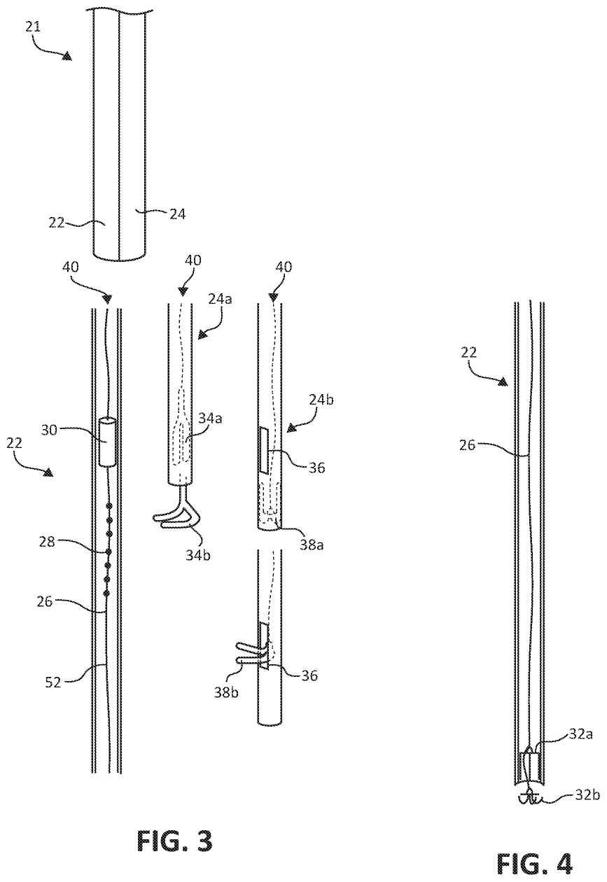

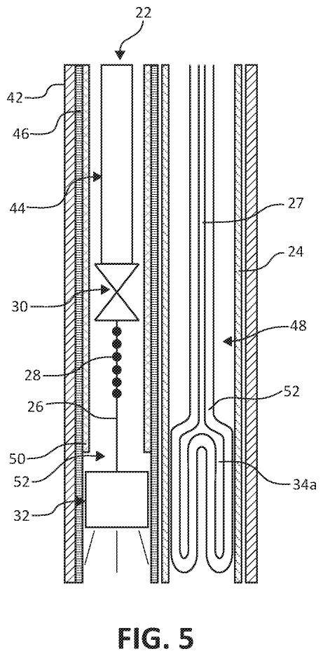

FIG. 2 is a conceptual schematic diagram illustrating an example tool system 20 that may be used, e.g., to add an artificial chordal attachment between a leaflet LF of a valve of heart 10 and tissue of the LV, such as the papillary muscle of the LV. FIG. 3 is a conceptual schematic diagram illustrating a magnified view of the portion of tool system 20 indicated in FIG. 2. FIG. 4 is a conceptual schematic diagram illustrating another magnified view of the portion of tool system 20 indicated in FIG. 2. FIG. 5 illustrates a conceptual schematic diagram illustrating another magnified view of the portion of tool system 20.

As shown, tool system 20 includes outer catheter 21 that acts as larger catheter containing two smaller catheters (first catheter 22 and second catheter 24 within outer catheter 21). First catheter 22 may be configured to, among others, puncture tissue such as a leaflet LF of MV of heart 10, and may be referred to as puncture catheter 22 in some cases. Second catheter may be configured to stabilize the leaflet LF of MV of heart 10, e.g., while first catheter 22 punctures the LF. In some examples, outer catheter 21 may define two inner lumens, one for puncture catheter 22 and one for stabilizer catheter 24. In other examples, however, first and second catheters 22, 24 may be positioned within the same lumen of outer catheter 21. In some examples, outer catheter 21 may allow for steering of tool system 20, e.g., along a trans-femoral, trans-septal pathway to allow for the addition of an artificial cordial attachment via a transcatheter process. For example, outer catheter 21 may include a braided nitinol shaft, which is flexible enough to be able to navigate tortuous anatomy during delivery/navigation to/through the septum and other anatomical locations. The distal portion of the nitinol braided shaft may be shape-set in the straight position, so that the portion puncturing through the leaflet LF and operating in the ventricle (to deliver anchor 32) is straight, but it's angle/orientation can be controlled about a proximal point (akin to a “pivot-point”), using a typical push/pull wire steering mechanism. As shown in the upper portion of FIG. 2, first catheter 22 and second catheter 24 may run adjacent to each other within outer catheter 21.

First catheter 22 defines first lumen 52 and may be used to deliver plug 30, line 26, and anchor 32 as described herein. Plug 30 and anchor 32 (labelled 32a in the undeployed position and labelled 32b in the deployed position) may be contained within first lumen 52 of first catheter 22. First line 26 extends within first lumen 52 and mechanically couples plug 30 to anchor 32. First line 26 may be any suitable material (e.g., a wire) such as neochord or polytetrafluoroethylene (PTFE) and may include a beaded section including one or more individual beads 28 (only a single bead is labelled in FIGS. 2-4). The bead(s) may be fixed to line 26. As described below, plug 30 may be slidable relative to line 26 and beads 28 to fix the length between plug 30 and anchor 32 when anchor 32 and plug 30 are secured in place within the leaflet LF and LV, respectively.

First catheter 22 includes hole puncture catheter 46 that is configured to puncture or otherwise make a hole (e.g., a through-hole) in leaflet LF, e.g., by exposing a sharp distal end out of first catheter 22 to the leaflet LF. Anchor deployment device 50 (also referred to as an anchor deployment lumen or anchor deployment lumen device) and plug stabilizer rod 44 are housed or otherwise contained within hole puncture catheter 46 as well as anchor 32, first line 26, and plug 30. Anchor deployment device 50 includes a lumen within hole puncture catheter 46 that pushes anchor 32 forward to deploy in LV tissue and also later retracts to deploy plug 30 in leaflet hole formed by hole puncture catheter 46. Plug stabilizing rod 44 is a rod that is configured to prevent plug 30 from moving upward (e.g., in a proximal direction towards a proximal end of outer catheter 21) when anchor deployment device 50 is retracted, until plug 30 is intentionally deployed at leaflet level within the hole formed in the leaflet.

Adjacent to first catheter 22 is second catheter 24 (stabilizer catheter) which defines second lumen 48. Second catheter 24 may be fixed axially relative to the overall catheter (e.g., fixed to outer catheter 21) or may be configured to rotate axially within the overall catheter (e.g., within a lumen of outer catheter 21 about a longitudinal axis of second catheter 24). Second line 27 (also referred to as a stabilizer wire or stabilizer line) is housed within second lumen 48 and is coupled to stabilizer member 34 (e.g., in the form of a foot) or stabilizer member 38 (which is an alternate example in the form of petals). Second catheter 24 is configured to stabilize leaflet LF (e.g., using stabilizer member 34 or 38) so that a hole can be punctured through leaflet tissue using hole puncture catheter 46. Each stabilizer member 34, 38 is configured to be held in a lower profile delivery configuration (e.g., straightened out) in a lumen of second catheter 24 until deployed by actuating second line 27 to take a stabilizer shape. When deployed from second catheter 24, stabilizer member 34, 38 may assume the stabilizer shape in which it includes portions that extend radially outward from a central longitudinal axis of second catheter 24. Stabilizer member 34, 38 may be formed from any suitable material, such as, but not limited to a shape memory material such as a shape-set nitinol wire having any suitable cross-sectional dimension (e.g., about 0.020 inch (about 0.5 millimeters)). Second line 27 may be a push/pull wire or rod in some examples.

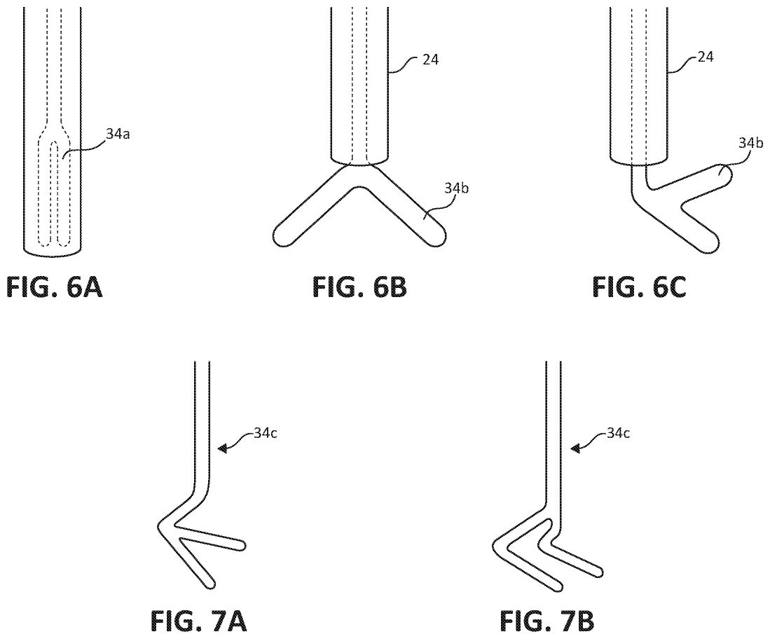

In some examples, stabilizer member 34 may have the design of a “foot” and/or “petal.” An example of the foot design is shown, e.g., in FIGS. 2 and 3 as stabilizer member 34a (undeployed position) and 34b (deployed position). In the case of a foot design, stabilizer member 34 may be deployed from bottom/distal end of second catheter 24. When deployed, stabilizer member 34b rests under leaflet LF and can be pulled up towards the leaflet LF to pull tissue of the leaflet taut. FIGS. 6A-6C show the foot design of stabilizer member 34 in further detail. As discussed above, stabilizer member 34a may be retractable into second catheter 24 in a contracted configuration (FIG. 6A). In a deployed configuration, stabilizer member 34b may include two arms defining an angle between about 45-degrees and about 180-degrees, such as about 90-degrees. Stabilizer 34b may be extended beyond a distal end of second catheter 24 (FIG. 6C) or abutted against the distal end of catheter 24 (FIG. 6B). In some examples, abutting stabilizer member 34b against the distal end of second catheter 24 may improve a stability of stabilizer member 34b. FIGS. 7A and 7B are alternative example of a foot design. As illustrated in FIG. 7A, in some examples, a portion of stabilizer member 34c proximal to the distal arms of stabilizer member 34c may include one or more deflections. Additionally, or alternatively, the distal arms of stabilizer member 34c may include one or more deflections. The deflections of stabilizer member 34c may be configured to improve a positioning of stabilizer member 34c, e.g., the distal arms thereof, relative to second catheter, a tissue, such as leaflet of a mitral valve, or both to improve capture or engagement of stabilizing member 34c with the tissue.

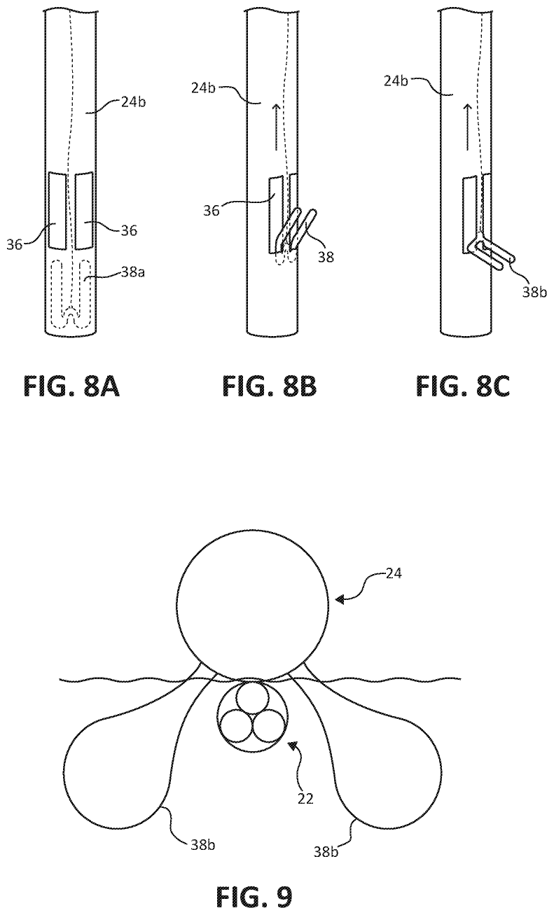

An example of the petal design is shown, e.g., in FIGS. 2 and 3 as stabilizer member 38a (undeployed position) and 38b (deployed position). As shown in FIG. 3, stabilizer member 38 may be deployed from side of second catheter 24 through aperture(s) 36, e.g., by pulling second line 27. When deployed, stabilizer member 38 is configured to rest under leaflet LF and pulled up to pull tissue taut. In some examples, apertures 36 are about 15 to about 45 degrees apart. FIGS. 8A-8C show the deployment of the petal design of stabilizer member 38 in further detail. As illustrated in FIG. 8A, when positioned distal to apertures 36, the petals of stabilizing member 38a are positioned within second catheter 24b. In some examples, the petals may include a preformed shape, e.g., by a preformed memory shape alloy or the like, to urge the petals toward a deployed configuration. For example, as stabilizing member 38 is drawn in the proximal direction, the petals will urge toward and out of aperture 36 (FIG. 8B), until fully deployed (FIG. 8C).

FIG. 9 is a conceptual diagram illustrating an example top view of stabilizer 38b of second catheter 24 deployed to stabilize the leaflet by engaging the tissue of leaflet. As shown, when second catheter 24 stabilizes the leaflet, first catheter 22 may be positioned over the leaflet so that puncture catheter 46 may be extended through the leaflet tissue.

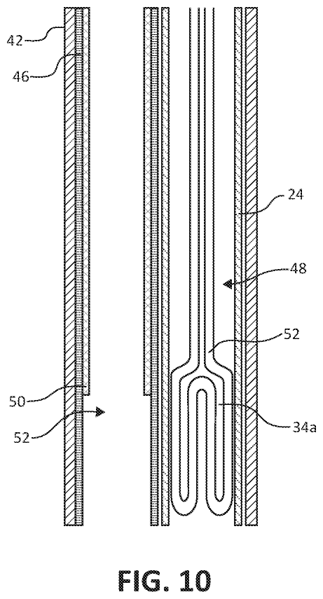

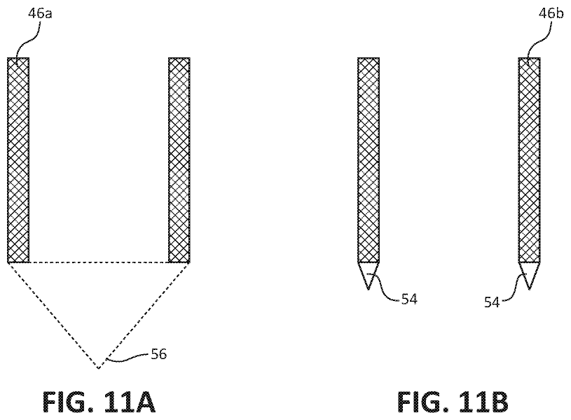

FIG. 10 is a conceptual diagram illustrating an example hole puncture catheter 46 along with a portion of tool system 20 in further detail. When used, hole puncture catheter 46 itself punctures and passes through the leaflet LF to deliver anchor 32 to the LV for attachment of anchor 32 to tissue of the LV. As described, stabilizer member 34 or 38 may engage tissue of the leaflet as hole puncture catheter punctures the leaflet. Hole puncture catheter 46 may then be retracted to deploy plug 30 in the leaflet hole and chordal length (the length of first line 26 between plug 30 and anchor 32) is adjusted through it. FIG. 11A shows one example of hole puncture catheter 46 with pointed tip attachment 56 at the distal end. For example, tip 56 may be configured to pierce a tissue, such as a leaflet of a valve. FIG. 11B shows another example of hole puncture catheter 46 with sharpened steel ring tip 54 at the distal end. For example, steel ring tip 54 may be configured to cut a circular shape hole in a tissue, such as a leaflet of a valve.

FIG. 12 is a conceptual diagram illustrating the position of anchor 32 within LV and the position of plug 30 near the leaflet surface. First line 26 connects plug 30 and anchor 32 and runs through the puncture hole in leaflet LF. For ease of illustration, not all of tool system 20 is shown in FIG. 12.

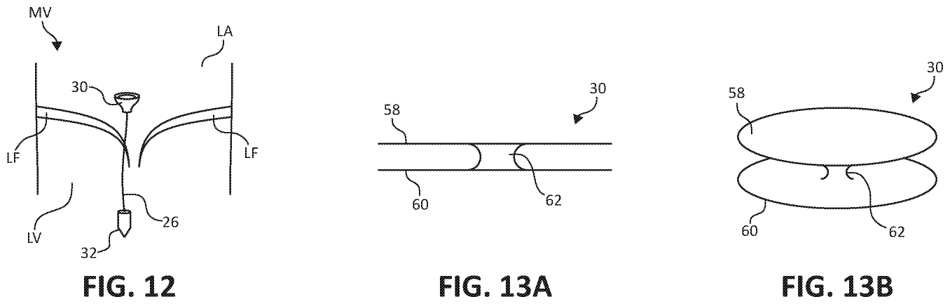

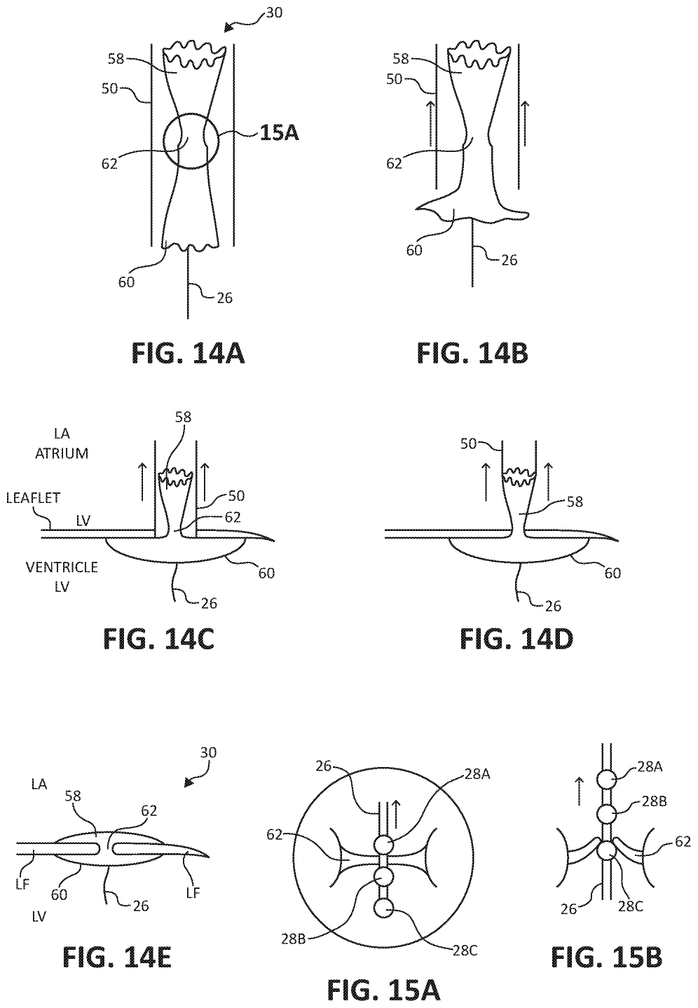

FIGS. 13A, 13B, and 14A-14E are conceptual diagrams illustrating details of plug 30. For example, plug 30 may include two opposing discs connected by the central pillar. In other examples, plug 30 may include other shapes, such as rectilinear shapes or shapes configured conform to a contour of an anatomical space. As illustrated in FIGS. 14A-14D, to attach plug 30 to the leaflet in the hole created by puncturing the leaflet (using hole puncture catheter 46), plug 30 is placed to seal the hole in the leaflet. Plug 30 includes two thin discs or “skirts”, one portion 58 which is configured to engage with (e.g., clings) one side of the leaflet LF, and one portion 60 configured to engage with to the other side of the leaflet LF. Portion 58 and portion 60 are mechanically connected by center portion 62. Plug 30 may be crimped/folded within first catheter 22 (e.g., as shown in FIGS. 13A and 14A until it is deployed by gradually retracting first catheter 22 (FIG. 14B) to allow the bottom “skirt” 60 to open below the leaflet LF (e.g., within LV) (FIG. 14C), then the top “skirt” 58 above the leaflet (FIG. 14D) until open on top of leadlet (FIG. 14E). Plug 30 may be any suitable material such as, e.g., a polymer, a pledget material, a tissue, or nitinol.

FIGS. 15A and 15B show the adjustment of first line 26 to adjust the length between anchor 32 and plug 30. As described, first line 26 includes one or more individual beads 26a-26c in a beaded segment. The beaded segment of first line 26 runs through center 62 of plug 20 at a “bridge” in center of plug 30, where the “bridge” is configured to flex in one direction (e.g., the proximal direction away from anchor 32) to allow beads through plug in one direction but not the other. By pulling first line 26 in the direction indicated in the figures, one or more bead 26a-26c may be pulled through plug 30 to shorten the length of first line 26 between anchor 32 and plug 30. In some examples, because of the one direction that beads 26a-26c may be pulled through plug 30, first line 26 may not be lengthened or otherwise loosened between anchor 32 and plug 30.

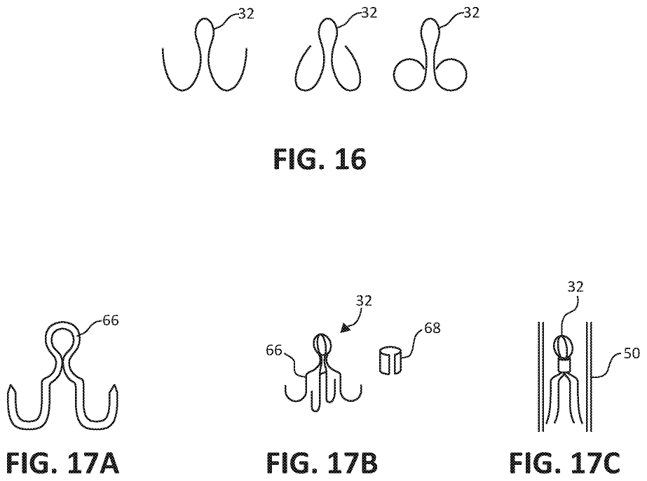

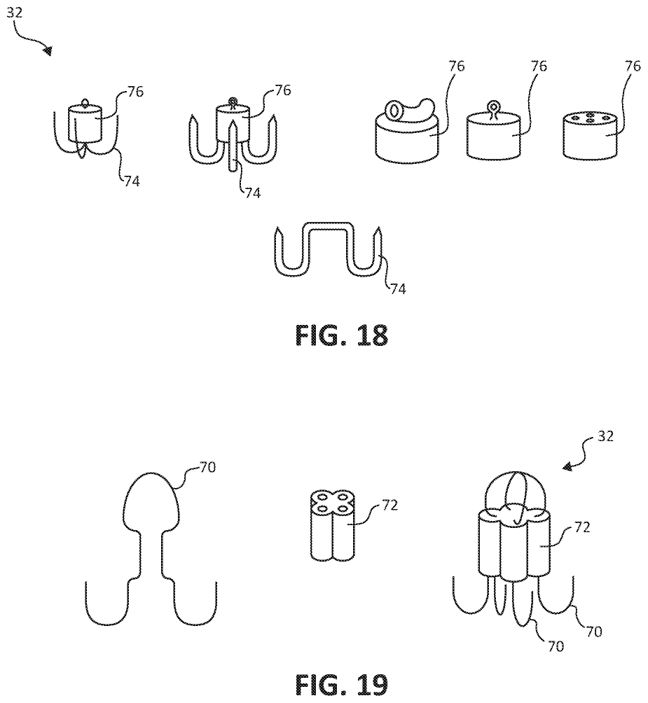

FIGS. 16, 17A-17C, 18, and 19 are conceptual diagrams illustrating examples of anchor 32 including possible wire shapes and connection mechanisms. In some examples, anchor 32 may include two shape memory (e.g., nitinol) shape-set pieces 66, 70, or 74, (e.g., 0.004 inch (about 0.10 millimeter) diameter nitinol wire) each containing two anchor prongs, which are then put together to create a four-pronged anchor. The two, two-pronged pieces 66, 70, or 74 may be held together & oriented at 90 degrees to each other using any suitable technique, e.g., a crimp 68, collar 72, or connective piece 76. Collar 72 may have through holes to receive the set pieces 70. Connective piece 76 may be a hollow cylinder with a loop on top and four holes on bottom. A flat segment of nitinol (NiTi) anchor (or other type anchor 32) may sit inside the cylinder on the bottom surface of cylinder & prongs protrude out of bottom holes.

Anchor 32 may be compressed (e.g., illustrated as 23A in FIG. 2) within anchor deployment device 50 until anchor 32 is deployed (e.g., illustrated as 32b in FIG. 2) out of the distal end of anchor deployment device 50 to attach to tissue of the LV. FIG. 16 illustrates example prong shapes of the pieces 66 of anchor 32. In some examples, anchor 32 includes three to four nitinol wire prongs (e.g., about 0.004 inch) connected at the top to first line 26. In other examples, anchor 32 may include any suitable number of prongs formed from any suitable material.

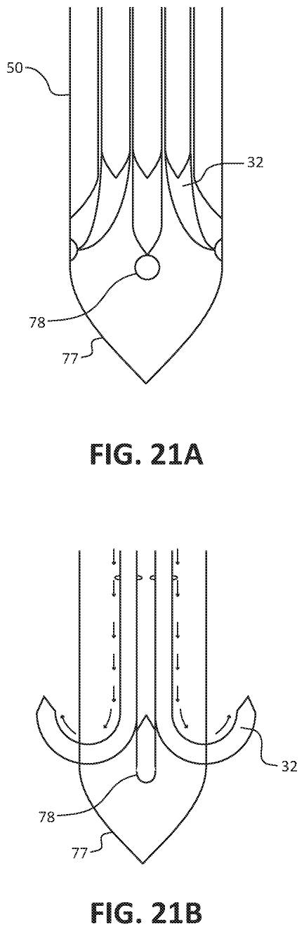

FIGS. 21A and 21B are conceptual diagrams illustrating examples of anchor 32 that may be used, e.g., to anchor an artificial chordal attachment to a tissue of the LV in accordance with some examples of the disclosure. As shown in FIG. 21a, anchor 32 may be in an undeployed position within anchor deployment device 50. Relatively large overall anchor tip 77 may be hollow and house, e.g., three to four straightened nitinol prongs/barbs. To attach anchor 32 to tissue, overall tip 77 may be driven into the LV tissue, (e.g., papillary muscle/LV wall) then barbs are advanced through holes 78 on overall tip, either passively by retraction of an outer sheath or actively, using a push rod/lumen from above, as shown in FIG. 21b. In some examples, the security of three to four anchor prongs, with safety of prongs being contained within one common tip until deployment in tissue may provide a benefit, e.g., several pronged anchor may provide secure attachment with relatively little adverse impact to the valve tissue due to the containment.

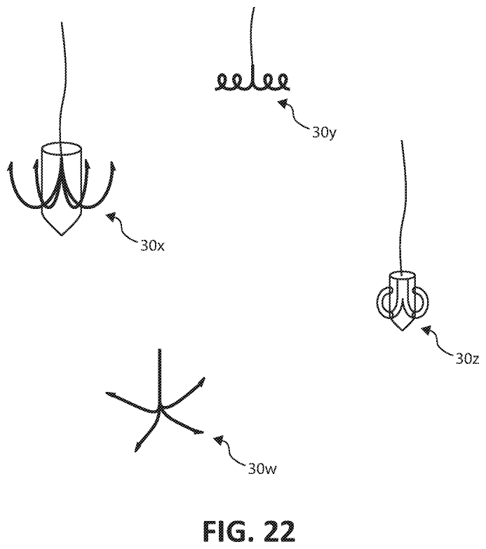

Anchor 32 is configured to engage with tissue in a heart of a patient, e.g., in the LV, in order to secure an end of first line 26 to the heart. FIG. 22 is a conceptual diagram illustrating other example shapes of anchors 30w-30z. Anchor 30w has a radial configuration. Anchor 30x has a hooking upward configuration. Anchor 30y has a corkscrew configuration. Anchor 30z has a configuration in which the prongs end hook back into the overall outer tip the anchor to “lock in” the anchor for added security to the tissue. Anchors of the disclosure may have four prongs or more or less than four prongs.

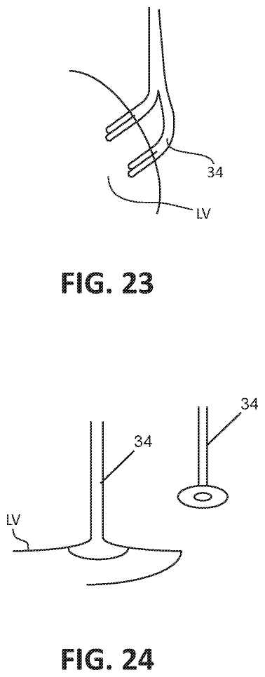

FIGS. 23 and 24 are conceptual diagrams illustrating other example designs of stabilizer member 34. In FIG. 23, stabilizer member 34 takes the form of a clip or clamp mechanism. In such an example, clip or clamp mechanism may grasp tissue of a LF to stabilize the LF to some extent before and/or during puncturing the LF. In FIG. 24, stabilizer member 34 takes the form of a suction member configured to engage the tissue of a LF via suction before and/or during puncturing the LF.

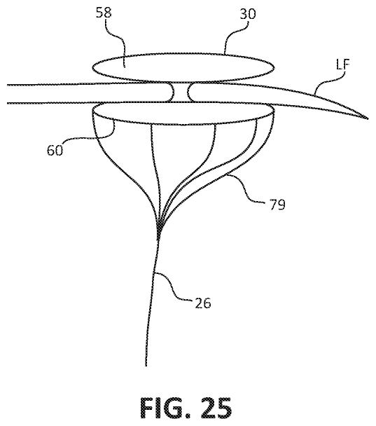

FIG. 25 is a conceptual diagram illustrating other example configurations for plug 30 and first line 26. As shown, multiple artificial chordae 79 may be coupled to the bottom portion 60 of plug 30 position within the hole/puncture in leaflet LF, which may connect to form a single artificial chordae/line 26 that attaches to anchor 32.

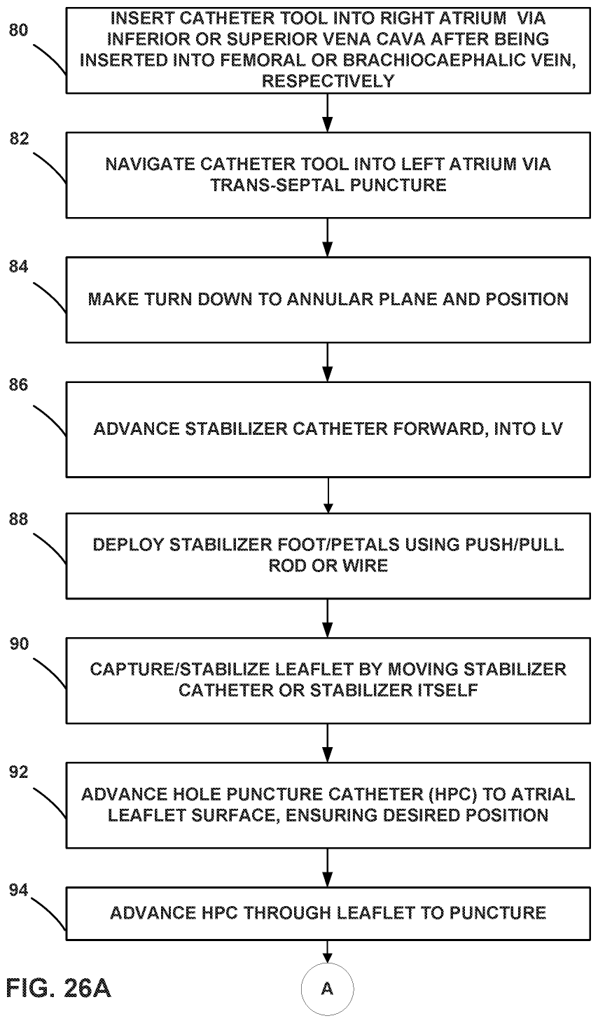

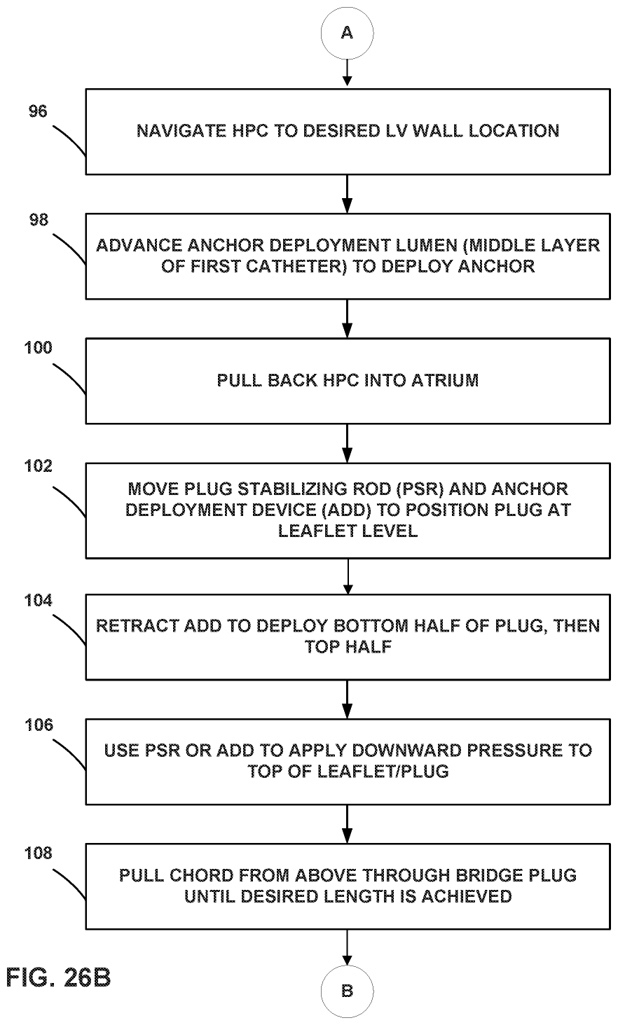

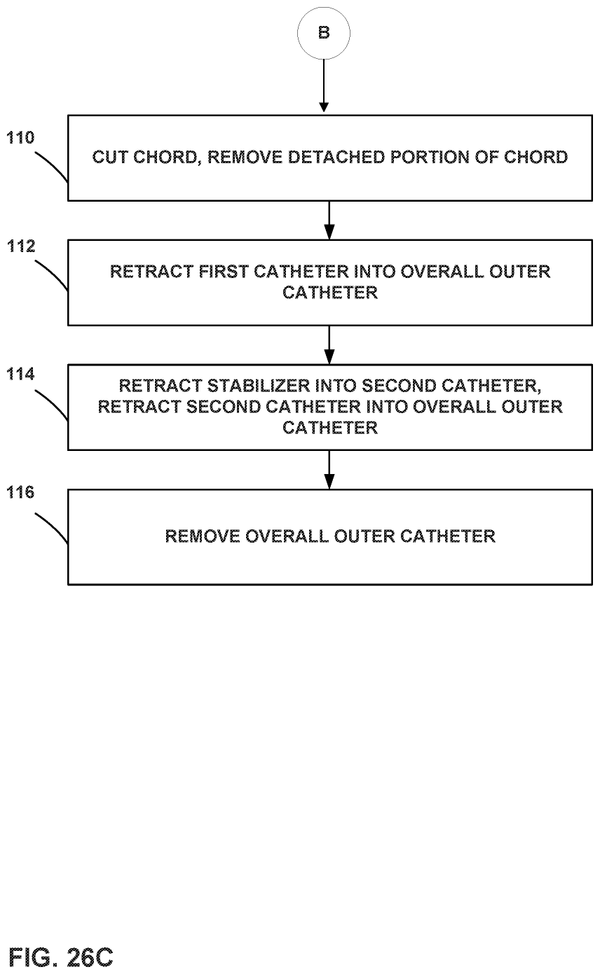

FIG. 26A-26C are flow diagrams illustrating an example technique for implanting an artificial chordal attachment between a leaflet LF of the MV and tissue of the LV of heart 10. For ease of description, the example technique is described with regard to tool system 20 although any suitable system may be used.

As shown in FIG. 26A, a distal portion of overall outer catheter 21 containing first and second catheter 22, 24 may be inserted into right atrium RA of heart 10 via the inferior or superior vena cava after being inserted into femoral or brachiocephalic vein, respectively (80). The distal portion of overall outer catheter 21 may then be navigated into the left atrium LA via trans-septal puncture (82). In this manner, the distal portion of tool system 20 may access the right atrium RA and then the left atrium LA through a trans-septal puncture. The distal portion of overall outer catheter 21 may then be turned down to the annular plane and position within the left atrium (84).

The distal portion of stabilizer catheter/second catheter 23 may then be advanced forward out of the distal end of overall outer catheter 21 into the left ventricle LV (86). Stabilizer foot 34 or petals 38 may be deployed by using the push/pull rod or wire (second line 27) within second catheter 24 (88). Stabilizer foot 32 or petals 38 may capture/engage/stabilize the leaflet LF by moving stabilizer/second catheter 24 and/or stabilizer foot 32 or petals 38 (90). When the leaflet is stabilized, hole puncture catheter 46 (HPC) may be advanced out of the overall outer catheter 21 to the atrial leaflet surface (92). Once the distal end of hole punch catheter 46 is at the desired location relative to on the leaflet LF, the sharpened tip of hole puncture catheter 46 may then be advance through the leaflet to puncture the leaflet LF (94). A distal portion of HPC 46 may be advanced through the puncture to navigate HPC 46 to a desired tissue location on the wall of the left ventricle LV (96). Anchor deployment device 50 may then be advanced to deploy anchor 32 out of the distal end and attached anchor 32 to the tissue (98). HPC 46 may be pulled back into the left atrium through the puncture in the leaflet (100).

Plug stabilizing rod (PSR) 44 and anchor deployment device (ADD) 50 may then be moved to position plug 30 at the leaflet level within the puncture (102). ADD 50 may be retracted to deploy the “bottom” half or portion 60 of plug 30 on the left ventricle side of the leaflet LF, and then further retracted to deploy the “top” half or portion 58 of plug 30 on the left atrium side of the leaflet LF (104). PSR 44 and/or ADD 50 may then be used to apply a downward/distal pressure to the top portion 58 of the leaflet and plug 30 (106). First line 26 may be pulled from above (proximally) to pull one or more of beads 28 through the bridge in center 62 of plug 30 until the desired length of first line 26 between plug 30 and anchor 32 is achieved (108). In some examples, the coaptation may be assessed (e.g., in real time using an echocardiogram or other technique).

First line 26 may then be cut above plug 30 and the proximal portion of first line 26 may be removed from first catheter 22 (110). First catheter 22 may be then retracted back into the overall outer catheter 21 (112). Stabilizer member 32 or 38 may be disengaged from the leaflet by retracting member 32 or 38 back into second catheter 24 (e.g., via second line 27) and second catheter 24 may be retracted back into the overall outer catheter 21 (114). Overall outer catheter 21 may then be removed from the patient (116).

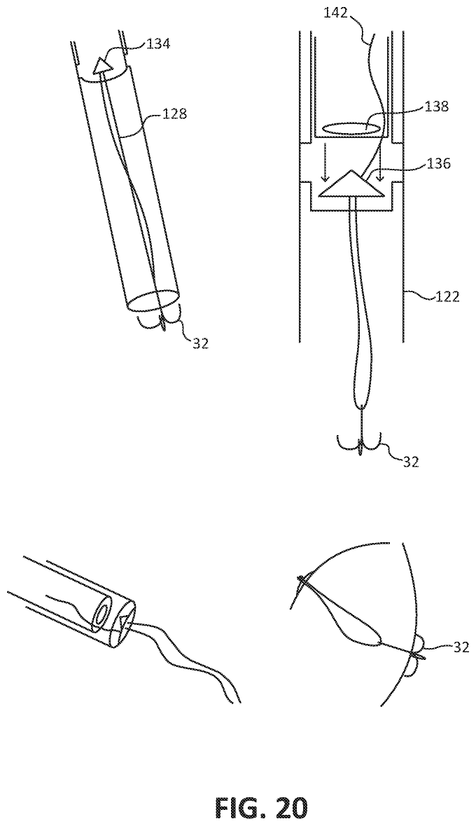

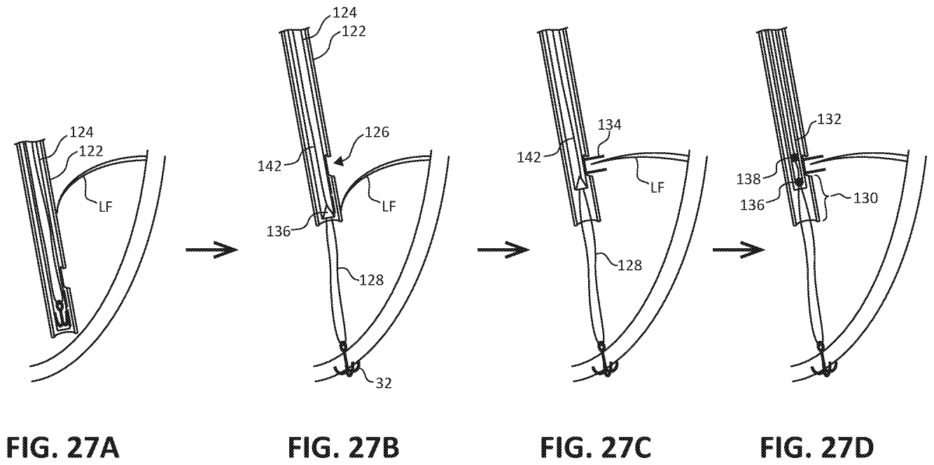

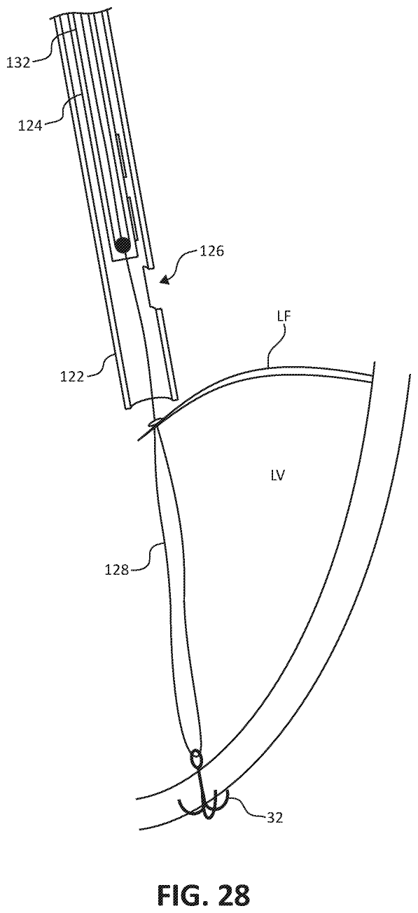

FIGS. 20 and 28 are conceptual diagram illustrating another example of a tool system used for an anchor first process for implanting an artificial chordal attachment between a leaflet of the MV and tissue of the LV. FIGS. 27A-27D are conceptual diagrams illustrating an example process for implanting an artificial chordal attachment between a leaflet of the MV and tissue of the LV using the described tool system.

As shown in each of FIGS. 20 and 27A through 28, the example tool system includes first catheter 122 and second catheter 124 within the inner lumen of first catheter 122. First catheter 122 may be introduced transseptally with second catheter 124 hosted inside. Both catheters 122, 124 may go into the LV and push out an anchor 32 such as anchor 32 described above (e.g., as in FIG. 17c). A suture 128 (or other material) is attached to that anchor and is hosted inside second catheter 124. One end of that “suture” is attached to the bottom of a sharp tip 136, the other end travels through the sharp tip 136 and is used to adjust the length of artificial chordal attachment between anchor 32 and sharp tip 126 before puncturing the leaflet LF. When anchor 32 is deployed, both first and second catheters 122, 124 may be pulled back. First catheter 122 has an opening 126 a certain distance from the distal tip of the first catheter 122. Opening 126 may be positioned near the free margin of the leaflet LF. Second catheter 124 may then be pulled up (or down) until leaflet stabilizers 134 actuate through opening 126 of first catheter 122. When leaflet stabilizers 134 activate, they leave an opening into the inner lumen of second catheter 124. The leaflet LF may be grabbed and the distal end of second catheter 124 may be positioned below the leaflet. The length of suture is determined and set (excess length removed). Third catheter 132 may then comes down, e.g., within second catheter 124, with a pledget-like device or other plug 138 and apply a force to the top of the leaflet LF to create force for the sharp tip to puncture through the leaflet LF (e.g., by pushing the leaflet surface against sharp tip 136) and become locked in the pledget 138. In this manner, the sharp tip 136 may puncture the leaflet LF and be attached to the leaflet, e.g., with or without the pledget 138. First catheter 122 may have a small, vertical slit running from the opening to the distal end of the first catheter 122, allowing the suture 128 to escape after full implantation. Similarly, there is a vertical slit on second catheter 124 from the level of the stabilizers to the distal end, allowing the removal of the delivery system after implantation.

In some examples, such a system and process may have a clip on the free margin of the leaflet instead of a “sharp tip” puncture. In both examples, the chord is attached to the mitral leaflet. In some examples, the steering required for such a procedure may be directed by first catheter 122. For example, pull wires on the outside of first catheter 122 may be offset from the opening in first catheter 122, and may allow for steering in two planes. In some examples, such a tool system may incorporate a guide sheath that stays in the atrium, or near the septum, that can provide additional steering.

The following clauses illustrate example subject matter described herein.

Clause 1. A catheter tool system comprising: a first catheter defining a first lumen; an anchor configured be located within the first lumen and to attach to a tissue of a left ventricle of a heart; a plug configured to be located within the first lumen and to attach to a leaflet of a mitral valve of the heart; a first line connecting the anchor to the plug within the first lumen; a second catheter defining a second lumen; a stabilizer member configured to be located within the second lumen and to extend out of the second lumen to stabilize the leaflet of the mitral valve relative to the second catheter; and a second line connected to the stabilizer member within the second lumen.

Clause 2. The system of clause 1, further comprising an outer catheter defining a third lumen, wherein the first catheter and second catheter are configured to be located within the third lumen of the outer catheter.

Clause 3. The system of clause 1 or 2, wherein a distal end of the first catheter is configured to puncture the leaflet of the mitral valve.

Clause 4. The system of clause 3, wherein the first catheter is configured to extend through the puncture in the leaflet of the mitral valve.

Clause 5. The system of any one of clauses 1 through 4, wherein the first line is slidable relative to the plug.

Clause 6. The system of clause 5, wherein the first line includes one or more beads, wherein the one or more beads are configured to slide through the plug and also engage the plug to fix a distance between the plug and the anchor.

Clause 7. The system of any one of clauses 1 through 6, wherein the anchor includes one or more prongs configured to engage a tissue of the left ventricle to connect the anchor to the left ventricle.

Clause 8. The system of any one of clauses 1 through 7, wherein the stabilizer member includes a foot configured to extend out an opening of the second lumen at or proximate to a distal end of the second catheter to engage the leaflet of the mitral valve.

Clause 9. The system of any one of clauses 1 through 8, wherein the second catheter define an opening at or proximate to a distal portion of the catheter, and wherein the stabilizer member includes a petal configured to extend out the opening of the second lumen at the distal portion of the second catheter to engage the leaflet of the mitral valve.

Clause 10. The system of any one of clauses 1 through 9, further comprising a plug stabilizer rod configured to be slidable within the first lumen of the first catheter to prevent movement of the plug in a proximal direction within the first lumen.

Clause 11. The system of any one of clauses 1 through 10, wherein the plug includes a first end portion and a second end portion, wherein the first end portion and the second end portion taper towards a center portion of the plug.

Clause 12. The system of clause 11, wherein, to connect the plug to the leaflet, the leaflet is positioned adjacent the center portion of the plug with the first portion of the plug on one side of the leaflet and the second portion of the plug on another side of the leaflet.

Clause 13. The system of any one of clauses 1 through 12, wherein the stabilizer member is configured to stabilize the leaflet of the mitral valve while the first catheter punctures the leaflet from a left atrium side to a left ventricle side.

Clause 14. The system of clause 13, wherein the anchor is configured to extend through the puncture and connect to the tissue of the left ventricle of the heart.

Clause 15. The system of clause 14, wherein the plug is configured to attach to the leaflet within the puncture while the anchor is connected to the tissue of the left ventricle of the heart.

Clause 16. The system of clause 15, wherein the stabilizer member is configured to disconnect from the leaflet while the anchor is connected to the tissue of the left ventricle of the heart and while the plug is attached to the leaflet.

Clause 17. A method comprising forming an artificial chordal attachment between a leaflet of a heart valve and an adjacent tissue using the tool system of any one of clauses 1 through 16.

Clause 18. The method of clause 17, wherein the heart valve comprises the mitral valve and the adjacent tissue comprises tissue of the left ventricle.

Clause 19. The method of clause 18, wherein the tissue of the left ventricle comprises a papillary muscle of the left ventricle.

Clause 20. The method of any one of clauses 17 through 19, wherein the artificial chordal attachment comprises the plug attached to the leaflet, the anchor attached to the adjacent tissue, and the first line connecting the anchor to the plug.

Clause 21. The method of clause 20, wherein forming the artificial chordal attachment comprises: puncturing the leaflet using the first catheter; inserting the first catheter through the puncture in the leaflet; attaching the anchor to the adjacent tissue using the first catheter; and attaching the plug to the leaflet within the puncture after the anchor has been attached to the adjacent tissue.

Clause 22. The method of clause 21, wherein puncturing the leaflet using the catheter tool system comprises puncturing the leaflet using a distal end of the first catheter.

Clause 23. The method of any one of clauses 21 or 22, further comprising stabilizing, prior to the puncturing of the leaflet using the first catheter, the leaflet relative to the second catheter using a stabilizer member of the second catheter.

Clause 24. The method of any one of clauses 21 through 23, further comprising adjusting a length of the first line between the anchor and the plug.

Clause 25. The method of clause 24, wherein adjusting the length of the first line between the anchor and the plug comprises adjusting the length of the first line between the anchor and the plug after the anchor has been attached to the adjacent tissue.

Clause 26. The method of clause 25, wherein adjusting the length of the first line between the anchor and the plug after the anchor has been attached to the adjacent tissue comprises adjusting the length of the first line between the anchor and the plug after the anchor has been attached to the adjacent tissue and after the plug has been attached to the puncture in the leaflet.

Clause 27. The method of any one of clause 24 through 26, wherein the adjusting the length of the first line between the anchor and the plug comprises pulling a bead coupled to the first line through the plug.

Clause 28. The method of any one of clauses 17 through 27, wherein forming the artificial chordal attachment between the leaflet of a heart valve and the adjacent tissue comprising accessing a left atrium of the heart with the catheter tool system via a trans-septum pathway.

Clause 29. A catheter tool system comprising: a first catheter defining a first lumen; an anchor configured be located within the first lumen and to attach to a tissue of a left ventricle of a heart; a second catheter within the first lumen and defining a second lumen; a stabilizer member configured to be located within the second lumen and to extend out of an opening in the first catheter to stabilize the leaflet of the mitral valve relative to the second catheter; a sharp tip member within the first lumen pointing away from a distal end of the first catheter; and a line connected to the anchor and the sharp tip member.

Clause 30. The system of clause 29, further comprising a third catheter within the second lumen, the third catheter including a plug at or near a distal end of the third catheter.

Clause 31. The system of clause 30, wherein the plug and sharp tip member are configured to be forced against each other to puncture a leaflet of a heart valve.

Clause 32. The system of any one of clauses 29 through 31, wherein the plug comprises a pledget-like member.

Clause 33. The system of any one of clauses 29 through 32, wherein the first catheter and the second catheter each include a vertical slit from a distal end to allow the line and anchor to be removed from the first catheter and second catheter.

Clause 34. A method comprising forming an artificial chordal attachment between a leaflet of a heart valve and an adjacent tissue using the tool system of any of the clauses 29-33.

Clause 35. The method of clause 34, wherein the heart valve comprises the mitral valve and the adjacent tissue comprises tissue of the left ventricle.

Clause 36. The method of clause 35, wherein the tissue of the left ventricle comprises a papillary muscle of the left ventricle.

Clause 37. The method of any one of clauses 34 through 36, wherein the artificial chordal attachment comprises the sharp tip attached to the leaflet, the anchor attached to the adjacent tissue, and the line connecting the anchor to the sharp tip.

Clause 38. The method of clause 37, wherein forming the artificial chordal attachment comprises: attaching the anchor to the adjacent tissue; engaging the leaflet using the stabilizers; puncturing the leaflet by applying a force between the plug and sharp tip using the third catheter; and removing the anchor and line through vertical slits in the first catheter and second catheter adjacent a distal end.

Various examples have been described. These and other examples are within the scope of the following claims.

What is claimed is:

1. A catheter tool system comprising:
a first catheter defining a first lumen;
an anchor configured be located within the first lumen and to attach to a tissue of a left ventricle of a heart;
a plug configured to be located within the first lumen and to attach to a leaflet of a mitral valve of the heart;
a first line connecting the anchor to the plug within the first lumen;
a second catheter defining a second lumen;
a stabilizer member configured to be located within the second lumen and to extend out of the second lumen to stabilize the leaflet of the mitral valve relative to the second catheter; and
a second line connected to the stabilizer member within the second lumen.

2. The system of claim 1, further comprising an outer catheter defining a third lumen, wherein the first catheter and second catheter are configured to be located within the third lumen of the outer catheter.

3. The system of claim 1, wherein a distal end of the first catheter is configured to puncture the leaflet of the mitral valve.

4. The system of claim 3, wherein the first catheter is configured to extend through the puncture in the leaflet of the mitral valve.

5. The system of claim 1, wherein the first line is slidable relative to the plug.

6. The system of claim 5, wherein the first line includes one or more beads, wherein the one or more beads are configured to slide through the plug and also engage the plug to fix a distance between the plug and the anchor.

7. The system of claim 1, wherein the anchor includes one or more prongs configured to engage a tissue of the left ventricle to connect the anchor to the left ventricle.

8. The system of claim 1, wherein the stabilizer member includes a foot configured to extend out an opening of the second lumen at or proximate to a distal end of the second catheter to engage the leaflet of the mitral valve.

9. The system of claim 1, wherein the second catheter define an opening at or proximate to a distal portion of the catheter, wherein the stabilizer member includes a petal configured to extend out the opening of the second lumen at the distal portion of the second catheter to engage the leaflet of the mitral valve.

10. The system of claim 1, further comprising a plug stabilizer rod configured to be slidable within the first lumen of the first catheter to prevent movement of the plug in a proximal direction within the first lumen.

11. The system of claim 1, wherein the plug includes a first end portion and a second end portion, wherein the first end portion and the second end portion taper towards a center portion of the plug.

12. The system of claim 11, wherein, to connect the plug to the leaflet, the leaflet is positioned adjacent the center portion of the plug with the first portion of the plug on one side of the leaflet and the second portion of the plug on another side of the leaflet.

13. The system of claim 1, wherein the stabilizer member is configured to stabilize the leaflet of the mitral valve while the first catheter punctures the leaflet from a left atrium side to a left ventricle side.

14. The system of claim 13, wherein the anchor is configured to extend through the puncture and connect to the tissue of the left ventricle of the heart.

15. The system of claim 14, wherein the plug is configured to attach to the leaflet within the puncture while the anchor is connected to the tissue of the left ventricle of the heart.

16. The system of claim 15, wherein the stabilizer member is configured to disconnect from the leaflet while the anchor is connected to the tissue of the left ventricle of the heart and while the plug is attached to the leaflet.

17. A method comprising forming an artificial chordal attachment between a leaflet of a heart valve and an adjacent tissue using a catheter tool system comprising:
a first catheter defining a first lumen;
an anchor configured be located within the first lumen and to attach to a tissue of a left ventricle of a heart;
a plug configured to be located within the first lumen and to attach to a leaflet of a mitral valve of the heart;
a first line connecting the anchor to the plug within the first lumen;
a second catheter defining a second lumen;
a stabilizer member configured to be located within the second lumen and to extend out of the second lumen to stabilize the leaflet of the mitral valve relative to the second catheter; and
a second line connected to the stabilizer member within the second lumen.

18. The method of claim 17, wherein forming the artificial chordal attachment comprises:
puncturing the leaflet using the first catheter;
inserting the first catheter through the puncture in the leaflet;
attaching the anchor to the adjacent tissue using the first catheter; and
attaching the plug to the leaflet within the puncture after the anchor has been attached to the adjacent tissue.

19. The method of claim 18, wherein puncturing the leaflet using the catheter tool system comprises puncturing the leaflet using a distal end of the first catheter.

20. A catheter tool system comprising:
a first catheter defining a first lumen;
an anchor configured be located within the first lumen and to attach to a tissue of a left ventricle of a heart;
a second catheter within the first lumen and defining a second lumen;
a stabilizer member configured to be located within the second lumen and to extend out of an opening in the first catheter to stabilize the leaflet of the mitral valve relative to the second catheter;
a sharp tip member within the first lumen pointing away from a distal end of the first catheter; and
a line connected to the anchor and the sharp tip member.
